(19) United States
(12) Patent Application Publication
(10) Pub. No.: US 2004/0077679 A1
(43) Pub. Date: Apr. 22, 2004

(54) Therapeutic treatment for the metabolic syndrome and type 2 diabetes

(76) Inventor: Anthony H. Cincotta, Tiverton, RI (US)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/627,014

(22) Filed: Jul. 25, 2003

Related U.S. Application Data
(60) Provisional application No. 60/399,180, filed on Jul. 29, 2002.

(51) Int. Cl. A61K 31/473; A61K 31/445

(52) U.S. Cl. 514/295; 514/317

(57) ABSTRACT

The present invention is directed to a method for treating the metabolic syndrome or Type 2 diabetes, comprising the step of administering to a subject in need of such treatment a pharmaceutical composition comprising (1) at least one compound that stimulates an increase in central dopaminergic neuronal activity level in the subject, and (2) at least one compound that stimulates a decrease in central noradrenergic neuronal activity level in the subject. The present invention is also directed to a method for treating the metabolic syndrome or Type 2 diabetes, comprising the step of administering to a subject in need of such treatment at least one compound that simultaneously stimulates (1) an increase in central dopaminergic neuronal activity level, and (2) a decrease in central noradrenergic neuronal activity level. The present invention is also directed to pharmaceutical compositions that include the above compounds and a pharmaceutically acceptable carrier.

### CROSS-REFERENCE TO RELATED APPLICATIONS

This application claims the benefit of U.S. Provisional Application No. 60/399,180 filed Jul. 29, 2002.

### BACKGROUND OF THE INVENTION

1. Field of the Invention

The present invention is directed to a method for treating the metabolic syndrome or Type 2 diabetes, and more particularly to a method for treating the metabolic syndrome or Type 2 diabetes by administering to a patient a pharmaceutical composition that increases the ratio of dopaminergic neuronal to noradrenergic neuronal activity within the hypothalamus of the central nervous system of the patient.

2. Brief Description of the Art

Obesity (commonly defined as a Body Mass Index of>30 kg/m²) is often associated with a variety of pathologic conditions such as hyperinsulinemia, insulin resistance, diabetes, hypertension, and dyslipidemia, and each of these conditions contributes to the risk of cardiovascular disease. Collectively, the pathologies that tend to associate (obesity, insulin resistance, dyslipidemia, and hypertension) have been termed “the metabolic syndrome” and are a major risk factor for cardiovascular disease, diabetes, and other diseases. The metabolic syndrome often predisposes one to overt Type 2 Diabetes.

A variety of treatments are available for diseases associated with obesity, including Type 2 Diabetes. For example, U.S. Pat. No. 6,506,799 discloses methods of treating cardiovascular diseases, dyslipidemia, dyslipoproteinemia, and hypertension comprising administering a composition comprising an ether compound.

U.S. Pat. No. 6,441,036 discloses fatty acid analogous which can be used for the treatment and/or prevention of obesity, fatty liver and hypertension.

U.S. Pat. No. 6,410,339 discloses use of cortisol agonist for preparing a system for diagnosis of the Metabolic Syndrome and related conditions as belly fatness, insulin resistance including increased risk of developing senile diabetes, i.e., diabetes type II, high blood fats and high blood pressure, in which system the dose of cortisol agonist is in an interval where a difference is obtained in the inhibitory effect of the autoproduction of cortisol in individuals suffering from the Metabolic Syndrome, compared to normal values.

U.S. Pat. No. 6,376,464 discloses peptides and peptide analogues that mimic the structural and pharmacological properties of human ApoA-I. The peptides and peptide analogues are useful to treat a variety of disorders associated with dyslipidemia.

U.S. Pat. No. 6,322,976 discloses, among other things, methods of diagnosing a disease associated with a defect in insulin action, glucose metabolism, fatty acid metabolism, and/or catecholamine action by detecting a mutation in the CD36 gene.

U.S. Pat. No. 6,197,765 discloses a treatment for metabolic syndrome (syndrome-X), and resulting complications, by administration of diazoxide.

U.S. Pat. No. 6,166,017 discloses a method for the medical treatment of diabetes mellitus type II and for counteracting the risk factors forming part of the Metabolic syndrome by administration of ketoconazole.

U.S. Pat. No. 6,040,292 discloses methods for the treatment of diabetes mellitus, including type I, type II, and insulin resistant diabetes (both type I and type II). The methods of the invention employ administration of rhIGF-I/IGFBP-3 complex to a subject suffering from the symptoms of diabetes mellitus. Administration of rhIGF-I/IGFBP-3 to a subject suffering from the symptoms of diabetes mellitus results in amelioration or stabilization of the symptoms of diabetes.

U.S. Pat. No. 5,877,183 discloses methods for the regulation and modification of lipid and glucose metabolism, but not metabolic syndrome, by administering to a subject a dopamine D1 agonist, optionally in combination with a dopamine D2 agonist, an alpha-1 adrenergic antagonist, an alpha-2 adrenergic agonist, or a serotonergic inhibitor, or optionally in combination with a dopamine D2 agonist coadministered with at least one of alpha-1 adrenergic antagonist, an alpha-2 adrenergic agonist, or a serotonergic inhibitor, and further administering the subject a serotonin 5HT_{lb} agonist. It is well known that dopamine agonists function to both activate and deactivate dopamine receptors and thereby reduce dopaminergic neuronal activity.

U.S. Pat. No. 5,741,503 discloses methods for regulating or ameliorating lipid metabolism which comprise administration or timed administration of inhibitors of dopamine beta hydroxylase (DBH). However, the focus of this technology is reduction in noradrenergic activity level only and does not increase dopaminergic neuronal activity inasmuch as DBH is not present in dopaminergic neurons that are anatomically distinct from noradrenergic neurons where DBH resides.

Neuronal activity appears to play a significant role in the metabolic syndrome diseases and Type 2 diabetes. However, there are few neuronal-based treatments for these diseases that consider both dopaminergic and noradrenergic neuronal activity. What is needed in the art are treatments for this disease that treat dopaminergic and noradrenergic neuronal activity simultaneously and in distinct ways. The present invention is believed to be an answer to that need.

### SUMMARY OF THE INVENTION

In one aspect, the present invention is directed to a method for treating the metabolic syndrome or Type 2 diabetes in a patient, comprising the step of increasing the ratio of dopaminergic neuronal to noradrenergic neuronal activity within the hypothalamus of the central nervous system of the patient.

In another aspect, the present invention is directed to a method for treating the metabolic syndrome or Type 2 diabetes, comprising the step of administering to a subject in need of such treatment a pharmaceutical composition comprising (1) at least one compound that stimulates an increase in central dopaminergic neuronal activity level in the subject, and (2) at least one compound that stimulates a decrease in central noradrenergic activity level in the subject.

In another aspect, the present invention is directed to a method for treating the metabolic syndrome or Type 2 diabetes, comprising the step of administering to a subject in need of such treatment at least one compound that simultaneously stimulates (1) an increase in central dopaminergic neuronal activity level, and (2) a decrease in central noradrenergic activity level.

In another aspect, the present invention is directed to a pharmaceutical composition effective for treating the metabolic syndrome or Type 2 diabetes, the composition comprising: (1) at least one central dopaminergic neuronal activity activator; (2) at least one central noradrenergic neuronal activity inhibitor; and (3) a pharmaceutically acceptable carrier.

In another aspect, the present invention is directed to a pharmaceutical composition effective for treating the metabolic syndrome or Type 2 diabetes, the composition comprising at least one compound that simultaneously stimulates (1) an increase in central dopaminergic neuronal activity level, and (2) a decrease in central noradrenergic neuronal activity level, the compound selected from the group consisting of catecholamine modifiers and a pharmaceutically acceptable carrier.

These and other aspect will be described in more detail in the following detailed description of the invention.

### DETAILED DESCRIPTION OF THE INVENTION

The novel treatment for the metabolic syndrome (obesity, insulin resistance, hyperlipidemia, and hypertension) and Type 2 diabetes consists of administering to a mammalian species in need of such treatment a pharmaceutical composition that simultaneously stimulates an increase in central dopaminergic neuronal activity level (particularly within neurons innervating the hypothalamus and the hypothalamus itself) and a decrease in central noradrenergic neuronal activity level (particularly within the brain stem region that innervates the hypothalamus and the hypothalamus itself). It has been unexpectedly discovered that increasing the ratio of dopaminergic neuronal to noradrenergic neuronal activity within the hypothalamus of the central nervous system improves the metabolic syndrome and/or type 2 diabetes conditions. As defined herein, “neuronal activity” refers to either an increase or decrease in the action potential of a neuron.

An important advantage of the present invention is avoidance of desensitization. Prior treatments result in the neuronal activity becoming “sensitized” to the application of drugs, and ultimately lead to ineffectiveness of these treatments. By contrast, the present invention avoids desensitization of stimulation of dopaminergic neurons or of inhibition of noradrenergic neurons, and thus makes the treatments highly effective.

In one embodiment, the method of the present invention includes administering to a subject in need of treatment for the metabolic syndrome or Type 2 diabetes a pharmaceutical composition comprising (1) at least one compound that stimulates an increase in central dopaminergic neuronal activity level in said subject, and (2) at least one compound that stimulates a decrease in central noradrenergic neuronal activity level in said subject. In an alternative embodiment, the pharmaceutical composition may include a single compound that stimulates an increase in central dopaminergic neuronal activity level as well as stimulates a decrease in central noradrenergic neuronal activity level. It is also contemplated that two, three, four, or more such compounds, each capable of simultaneously stimulating an increase in central dopaminergic neuronal activity level as well as stimulates a decrease in central noradrenergic neuronal activity level, may be used in the pharmaceutical composition. In all embodiments, however, the ratio of dopaminergic neuronal to noradrenergic neuronal activity within the hypothalamus is increased.

The increase in central dopaminergic neuronal activity level can take place by any mechanism. In preferred embodiments, the increase in central dopaminergic neuronal activity level occurs by including in the pharmaceutical composition at least one compound that stimulates an increase in central dopaminergic neuronal activity level. Preferably, such compounds include, but are not limited to, dopamine reuptake inhibitors, dopamine presynaptic transporter inhibitors, presynaptic dopamine release enhancers, post synaptic dopamine receptor agonists, dopamine synthesis stimulators, and/or dopamine catabolism inhibitors. Examples of useful compounds that stimulate an increase in central dopaminergic neuronal activity level include, but are not limited to, GBR-12935 (known as 1-[2-(diphenylmethoxy)ethyl]-4-(3-phenylpropyl)piperazine); BDNF (Brain Derived Neurotrophic Factor), quinpirole ((4aR-trans)-4,4a,5,6,7,8,8a,9-octahydro-5-propyl-1H-pyrazolo[3,4-g]quinoline); SKF3 8393 (1-phenyl-7,8-dihydroxy-2,3,4,5-tetrahydro-1H-3-benzazepine hydrochloride); deprenyl (also known as “Selegiline”); apomorphine, pramipexole (sold commercially under the name “Mirapex”), GBR-12909 (“Vanoxerine”, 1-2-(bis(4-fluorophenyl)-methoxy)-ethyl-4-(3-phenylpropyl) piperazine); and combinations thereof.

The inhibition of noradrenergic neuronal activities may also be accomplished via any mechanism. In preferred embodiments, stimulation of a decrease in central noradrenergic activity level occurs by administration of at least one compound that results in a decrease in central noradrenergic activity level. Preferably, such compounds include, but are not limited to, postsynaptic noradrenergic receptor blockade compounds, inhibitors of noradrenalin release, inhibitors of noradrenalin synthesis, activators of noradrenalin presynaptic reuptake, and activators of noradrenalin catabolism presynaptically and in the synapse. Examples of useful compounds that decrease central noradrenergic activity level include, but are not limited to, prazosin (1-(4-amino-6,7-dimethoxy-2-quinazolinyl)-4-(2-furanylcarbonyl)piperizin propranolol (1(isopropylamino)-3-(1-naphthyloxy)-2-propanol); clonidine (2-(2,6-dichloroanilino)-2-imidazoline); fusaric acid (5-butyl-2-pyridinecarboxylic acid; 5-butylpicolinic acid); dopamine; phenoxybenzamine; phentolamine, (3-[[(4,5-dihydro-1H-imidazol-2-yl)methyl](4-methylphenyl) amino]phenol; 2-[N-(m-hydroxyphenyl-p-toluidineomethyl)imidazoline); guanfacine (sold under the brand name “Tenex”); and combinations thereof.

As indicated above, the method of the invention may also include administration of a pharmaceutical composition that includes a single or individual compound that simultaneously stimulates an increase in central dopaminergic neuronal activity level and a decrease in central noradrenergic neuronal activity level. Examples of such compounds include catecholamine modifiers, such as dopamine.

The compounds of the invention are preferably administered internally, e.g., orally or intravenously, in the form of conventional pharmaceutical compositions, for example in conventional enteral or parenteral pharmaceutically acceptable excipients containing organic and/or inorganic inert carriers, such as water, gelatin, lactose, starch, magnesium stearate, talc, plant oils, gums, alcohol, Vaseline, or the like. The pharmaceutical compositions can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical compositions may also contain other therapeutically active materials. The pharmaceutical compositions of the invention can be made using conventional methods know in the art of pharmaceutical manufacturing.

The pharmaceutical compositions of the invention should include an amount of the compound(s) of the invention effective for treatment of the metabolic syndrome or Type 2 diabetes. The effective dosage will depend on the severity of the diseases and the activity of the particular compound(s) employed, and is thus within the ordinary skill of the art to determine for any particular host mammal or other host organism. Suitable dosages may be, for example, in the range of about 0.1 to about 100 mg per kg for a human being, and more preferably from about 2 to about 50 mg per kg for a human being.

The ratio of the compound(s) that stimulates an increase in central dopaminergic neuronal activity level to the compound(s) that stimulates a decrease in central noradrenergic neuronal activity level in the pharmaceutical composition generally ranges from about 500:1 to 1:500 on a weight-to-weight basis (w:w), and more preferably from about 100:1 to 1:100 on a weight-to-weight basis (w:w).

The present invention is further described in detail by means of the following Example. All parts and percentages are by weight unless explicitly stated otherwise.

### EXAMPLE

Four different groups of animals exhibiting the metabolic syndrome and/or Type 2 diabetes are treated with either saline as control, central dopamine neuronal activity activator(s), central noradrenergic neuronal activity inhibitor(s), or a molecular entity or entities that is/are both a central dopaminergic neuronal activity activator and central noradrenergic neuronal activity inhibitor, respectively.

Relative to the control group the dopaminergic neuronal activator/noradrenergic neuronal activity inhibitor group exhibits the greatest improvement in metabolism (decrease in obesity, dyslipidemia, hypertension, insulin resistance, hyperinsulinemia, and/or hyperglycemia) that is also significantly better than that of either the dopaminergic activator or noradrenergic inhibitor groups. An unexpected synergism between the dopaminergic neuronal activity stimulator(s) and noradrenergic neuronal activity inhibitors(s) is observed relative to the effects on improvement of the metabolic syndrome and/ or type 2 diabetes.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A method for treating the metabolic syndrome or Type 2 diabetes in a patient, comprising the step of increasing the ratio of dopaminergic neuronal to noradrenergic neuronal activity within the hypothalamus of the central nervous system of said patient.

2. A method for treating the metabolic syndrome or Type 2 diabetes, comprising the step of:
administering to a subject in need of such treatment a pharmaceutical composition comprising (1) at least one compound that stimulates an increase in central dopaminergic neuronal activity level in said subject, and (2) at least one compound that stimulates a decrease in central noradrenergic neuronal activity level in said subject.

3. The method of claim 2, wherein said increase in central dopaminergic neuronal activity level occurs within neurons innervating the hypothalamus and the hypothalamus itself.

4. The method of claim 2, wherein said at least one compound that stimulates an increase in central dopaminergic neuronal activity level is selected from the group consisting of dopamine reuptake inhibitor compounds, dopamine presynaptic transporter inhibitor compounds, presynaptic dopamine release enhancer compounds, post synaptic dopamine receptor agonist compounds, dopamine synthesis stimulator compounds, dopamine catabolism inhibitor compounds, and combinations thereof.

5. The method of claim 2, wherein said at least one compound that stimulates an increase in central dopaminergic neuronal activity level is selected from the group consisting of GBR-12935, BDNF, quinpirole, SKF38393, deprenyl, apomorphine, pramipexole, GBR-12909, and combinations thereof.

6. The method of claim 2, wherein said decrease in central noradrenergic neuronal activity level occurs within the brain stem region that innervates the hypothalamus and the hypothalamus itself.

7. The method of claim 2, wherein said at least one compound that stimulates a decrease in central noradrenergic neuronal activity level is selected from the group consisting of postsynaptic noradrenergic receptor blockade compounds, inhibitors of noradrenalin release, inhibitors of noradrenalin synthesis, activators of noradrenalin presynaptic reuptake, and activators of noradrenalin catabolism presynaptically and in the synapse, and combinations thereof.

8. The method of claim 2, wherein said at least one compound that stimulates a decrease in central noradrenergic neuronal activity level is selected from the group consisting of prazosin, propranolol, clonidine, fusaric acid, dopamine, phenoxybenzamine, phentolamine, guanfacine, and combinations thereof.

9. The method of claim 2, wherein the ratio of said at least one compound that stimulates an increase in central dopaminergic neuronal activity level to said at least one compound that stimulates a decrease in central noradrenergic neuronal activity level in said pharmaceutical composition ranges from about 500:1 to 1:500 on a weight-to-weight (w:w) basis.

10. The method of claim 2, wherein the ratio of said at least one compound that stimulates an increase in central dopaminergic neuronal activity level to said at least one compound that stimulates a decrease in central noradrenergic activity level in said pharmaceutical composition ranges from about 100:1 to 1:100 on a weight-to-weight (w:w) basis.

11. A method for treating the metabolic syndrome or Type 2 diabetes, comprising the step of:
administering to a subject in need of such treatment a pharmaceutical composition comprising at least one compound that simultaneously stimulates (1) an increase in central dopaminergic neuronal activity level, and (2) a decrease in central noradrenergic neuronal activity level.

12. The method of claim 11, wherein said increase in central dopaminergic neuronal activity level occurs within neurons innervating the hypothalamus and the hypothalamus itself.

13. The method of claim 11, wherein said decrease in central noradrenergic neuronal activity level occurs within the brain stem region that innervates the hypothalamus and the hypothalamus itself.

14. The method of claim 11, wherein said compound is selected from the group consisting of catecholamine modifiers.

15. A pharmaceutical composition effective for treating the metabolic syndrome or Type 2 diabetes, said composition comprising:
(1) at least one central dopaminergic neuronal activity activator;
(2) at least one central noradrenergic neuronal activity inhibitor; and
(3) a pharmaceutically acceptable carrier.

16. The pharmaceutical composition of claim 15, wherein said at least one central dopaminergic neuronal activity activator is selected from the group consisting of GBR-12935, BDNF, quinpirole, SKF38393, deprenyl, apomorphine, pramipexole, GBR12909, and combinations thereof.

17. The pharmaceutical composition of claim 15, wherein said at least one central noradrenergic neuronal activity inhibitor is selected from the group consisting of prazosin, propranolol, clonidine, fusaric acid, dopamine, phenoxybenzamine, phentolamine, guanfacine, and combinations thereof.

18. The pharmaceutical composition of claim 15, wherein the ratio of said at least one central dopaminergic neuronal activity activator to said at least one central noradrenergic neuronal activity inhibitor ranges from about 500:1 to 1:500 on a weight-to-weight (w:w) basis.

19. The pharmaceutical composition of claim 15, wherein the ratio of said at least one central dopaminergic neuronal activity activator to said at least one central noradrenergic neuronal activity inhibitor ranges from about 100:1 to 1:100 on a weight-to-weight (w:w) basis.

20. A pharmaceutical composition effective for treating the metabolic syndrome or Type 2 diabetes, said composition comprising
at least one compound that simultaneously stimulates (1) an increase in central dopaminergic neuronal activity level, and (2) a decrease in central noradrenergic neuronal activity level, said compound selected from the group consisting of catecholamine modifiers and
a pharmaceutically acceptable carrier.
